(19) United States
(12) Patent Application Publication
(10) Pub. No.: US 2018/0153730 A1
(43) Pub. Date: Jun. 7, 2018

(54) SECONDARY BAG FOR URINE

(71) Applicants: Alessandro DI TEODORO, Pescara PE (IT); Antonio FOLCANDO, Foggia FG (IT); Evoluzione S.r.l., Roma RM (IT); Servimed S.r.l., Roma RM (IT)

(72) Inventors: Ettore FOLCANDO, Pescara PE (IT); Alessandro Dl TEODORO, Pescara PE (IT); Francesco NAPOLITANO, Roma RM (IT); Enrico DE LUTIO, Roma RM (IT)

(21) Appl. No.: 15/568,379

(22) PCT Filed: Sep. 11, 2015
(86) PCT No.: PCT/IB2015/056965

§ 371 Date: Oct. 20, 2017

(30) Foreign Application Priority Data
Apr. 23, 2015 (IT) 102015902346192

(51) Int. Cl. A61F 5/44; A61F 5/441

(52) U.S. Cl. CPC A61F 5/4401; A61F 5/441; A61F 5/4404

(57) ABSTRACT

The present invention relates to a bag apt to be connected to the primary bags in turn connected to a catheter so as to perform safely the emptying of the bag, by eliminating all risks connected to the accidental spilling of liquids.

### FIELD OF THE INVENTION

The present invention relates to a bag apt to be connected to the primary bags in turn connected to a catheter so as to perform safely the emptying of the bag, by eliminating all risks connected to the accidental spilling of the liquids.

### STATE OF ART

Currently in the hospitalization days many patients are catheterized, that is they are provided with a small tube made of lattice or silicone which is introduced into the bladder through urethra to favour the discharge of urine outside. The main problem linked to the use of catheters is that the bags connected to the catheter, once they have filled up, have to be emptied to be disposed of.

The risks connected to the handling of such bags, in particular in the emptying step, force the operators to provide themselves with personal protection equipment to lower the infection risk during this process.

The object of the present invention is to solve this problem by providing a bag for urine according to claim 1.

Additional features of the bag for urine of the subject invention are defined in the corresponding depending claims.

The present invention, by overcoming the mentioned problems of the known art, involves several and evident advantages:

removal of contamination risk for the operator caused by splashes during emptying, since such procedure is no more necessary thanks to the fact that the bag is disposed of as solid waste. Consequently, the operator needs no more to provide himself/herself with personal protection equipment as he/she acts under maximum safety conditions.

There are no more risks of accidental spillages potentially dangerous for whoever is in proximity.

The sachet of water-soluble material existing in the bag, as soon as it comes in contact with urine, dissolves quickly by allowing the molecule super absorbent to transform into solid substance the urine coming from the main bag. In this way it is avoided that the content of the same remains liquid and then more subjected to risks of accidental spillage.

To support what mentioned above, an additional thing to be added is that the waste produced after gelification is by now a solid waste, therefore less onerous to be handled and above all less dangerous (as without risks due to possible splashes).

Other advantages, as well as the features and the use modes of the present invention will result evident from the following detailed description of some preferred embodiments, shown by way of example and not for limitative purposes, by making reference to the figures of the enclosed drawings, wherein:

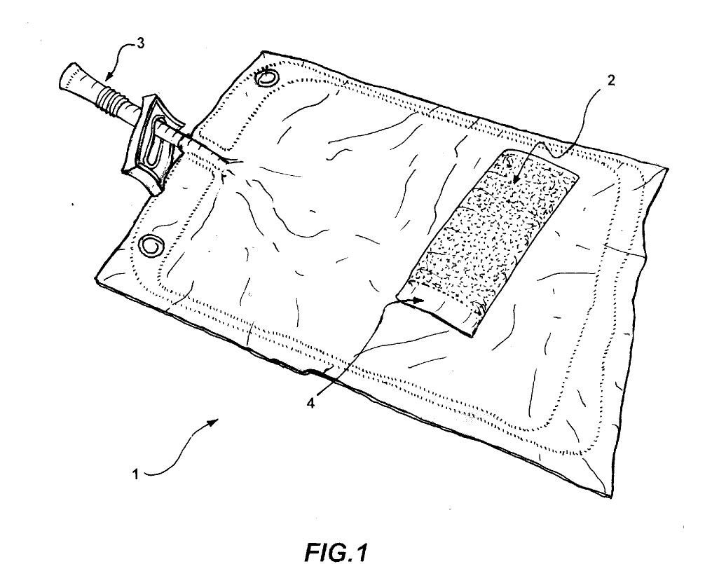

FIG. 1 is an overall view of the bag according to a preferred embodiment of the present invention.

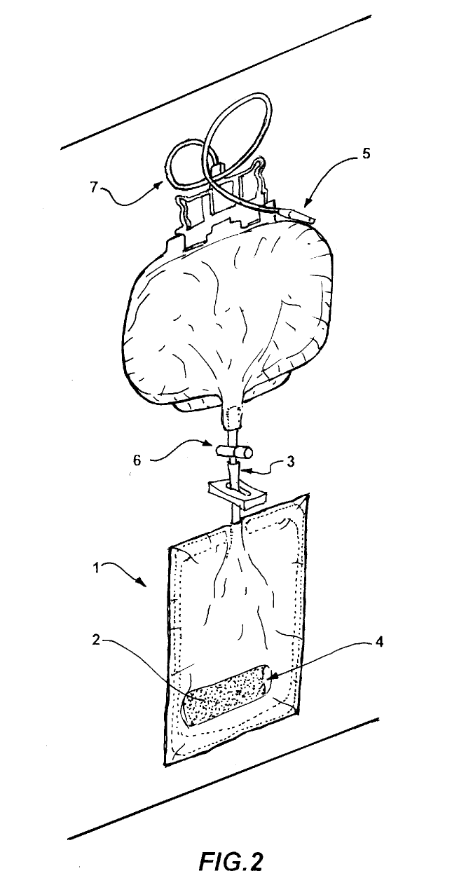

FIG. 2 is an overall view of a kit used according to a preferred embodiment of the present invention.

The present invention will be described hereinafter in details by making reference to the above-mentioned figures.

By firstly making reference to FIG. 1, this represents an overall view showing, as a whole, a bag 1 according to the present invention, according to a preferred embodiment.

The secondary bag of the present invention is a bag suitable to include liquid organic waste, in particular urine, and it is characterized in that it includes inside thereof a water soluble bag 4 comprising inside thereof a substance 2 apt to absorb urine. The secondary bag 1 further comprises means 3 apt to connect said secondary bag 1 to a primary bag 5 whereby the urine contained in said primary bag 5 is emptied into said secondary bag 1.

In the present description under the term primary bag 5 any bag is meant suitable to include liquid organic waste, in particular urine, connected or connectable to a catheter 7 and comprising means 6 for emptying the primary bag, for example standard connectors known to the person filled in the art currently used for emptying this type of bags.

In the present description under the term secondary bag 1 any bag is meant suitable to include liquid organic waste, in particular urine, comprising means 3 apt to connect said secondary bag 1 to a primary bag 5 whereby when the primary bag 5 is filled up with urine, the urine included in the primary bag 5 can be emptied into the secondary bag 1. Said means 3, for example, comprises a universal connector connectable to means 6 for emptying the primary bags 5.

Example of substances suitable to absorb the urine are super absorbent powders with absorption capacity between 60 and 500 times the weight of the liquids, such as for example the cross-linked sodium polyacrylate, preferably provided with system aromatizer, for example the one available under the trade name of Sgaiel. Such powders are able to transform the urine into gel. Under provided with “system aromatizer” it is meant that the substance absorbent can be itself perfumed or be mixed to an additional perfumed substance.

A subject of the present invention is even a kit comprising one or more primary bags according to the present invention and at least a primary bag 5. According to an embodiment, such kit comprises one or more catheters 7 connected or connectable to said primary bag 5. Preferably the catheter 7 will be connected to the primary bag 5 in opposite position with respect to the emptying means 6.

In the light of the above-illustrated description it appears clear that the operation of the bag and of the subject kit is extremely easy as it is sufficient that the operator connects the secondary bag to the primary bag which he/she has to empty and during the filling-up thereof the molecule will enter in function automatically thanks to the melting of the water-soluble sack.

The present invention has been so far described by referring to some embodiments thereof. It is to be meant that other embodiments belonging to the same inventive core may exist, all within the protective scope of the herebelow reported claims.

1. A secondary bag for urine comprising a connector to connect said secondary bag to a primary bag whereby the urine contained in said primary bag is emptied into said secondary bag and wherein said secondary bag contains a water soluble bag containing a substance apt to absorb urine, wherein said substance comprises an absorbent molecule adapted to transform the urine into gel.

2. The secondary bag according to claim 1, wherein said substance is comprises an absorbent powder with absorption capacity between 60 and 500 times a weight of a liquid to absorb.

3. The secondary bag according to claim 1, wherein said substance comprises a crosslinked sodium polyacrylate.

4. The secondary bag according to claim 1, wherein said substance comprises a system aromatizer.

5. The secondary bag according to claim 1, wherein the secondary bag comprises at least one of polyethylene, biodegradable polyethylene, or pvc.

6. The secondary bag according to claim 1, wherein said connector comprises a universal connector connectable to an emptying connector of said primary bag.

7. The secondary bag according to claim 1, wherein said primary bag is filled with urine via a catheter connected or connectable to said primary bag.

8. A Kit comprising one or more secondary bags according to claim 1 and at least one primary bag.

9. The Kit according to claim 8, further comprising one or more catheters connected or connectable to said primary bag.

10. (canceled)
